(19) United States
(12) Patent Application Publication
(10) Pub. No.: US 2022/0248957 A1
(43) Pub. Date: Aug. 11, 2022

(54) Remote Patient Medical Evaluation Systems and Methods

(71) Applicant: Abdullalbrahim ABDULWAHEED, Holbrook, MA (US)

(72) Inventor: Abdullalbrahim ABDULWAHEED, Holbrook, MA (US)

(21) Appl. No.: 17/168,607

(22) Filed: Feb. 5, 2021

(51) Int. Cl. A61B 5/00; A61B 5/01; G16H 80/00

(52) U.S. Cl. CPC A61B 5/0022; A61B 5/015; A61B 5/0013; G16H 80/00; A61B 5/7435; A61B 5/0008; A61B 5/0046

(57) ABSTRACT

A method for remote medical evaluation of a patient includes providing a patient-side application to a patient platform and a practitioner-side application to a practitioner platform, establishing an electronic communication connection between the patient platform and the practitioner platform, transmitting electronic command signals from the practitioner platform to the patient platform to remotely control one or more components of the patient platform, receiving at the practitioner platform information obtained from operation of the one or more remotely controlled patient platform components, and providing on the practitioner platform the obtained information to a medical practitioner. A non-transitory computer readable medium and a system to implement the method are also disclosed.

### FIELD OF THE INVENTION

The present invention relates to systems and methods for remote evaluation of a patient by a medical professional. More specifically, to systems and methods for providing a real-time, interactive platform for patient evaluation, diagnosis, and communication.

### BACKGROUND

Conventional remote patient monitoring can use various technologies that collect patient data and transmit that data to an external location for storage. Commercially available wearable medical devices can be equipped with sensors to noninvasively monitor a user's physical condition (e.g., heart rate, blood pressure, respiratory rate, sleep patterns, blood oxygen, blood glucose, etc.) Usually requiring direction from a medical professional, implantable medical devices are inserted into the body (e.g., pacemaker, defibrillator, electrocardiogram recorder, medication infusion devices, etc.). The stored monitored data provides a snapshot of a patient's status at some prior point in time.

### BRIEF DESCRIPTION OF THE DRAWINGS

FIG. 1 depicts a remote patient medical evaluation system in accordance with embodiments;

FIGS. 2A-2C depict a process for conducting a remote patient medical evaluation in accordance with embodiments;

FIG. 3 depicts a patient view of an interactive display in accordance with embodiments;

FIGS. 4A-4G depict various practitioner views of an interactive display in accordance with embodiments; and

FIG. 5 depicts a view of an interactive display accessible to both the patient and the practitioner in accordance with embodiments.

### DETAILED DESCRIPTION

Embodying systems and methods provide a medical practitioner (e.g., doctor, dentist, podiatrist, physician's assistant, nurse practitioner, etc.) the ability to evaluate and/or diagnosis a patient. Information on the patient's condition(s) can be remotely accessed in about real-time (i.e., at the time of observance). This information can be used to evaluate and diagnosis the patient's condition. Embodiments provide interactive audio and video communication between the patient and practitioner to assist in the execution of various diagnostic protocol processes implemented by the practitioner remote from the patient. In some embodiments, data from a patient's wearable and/or implanted device(s) can be communicatively linked for display in real-time to the practitioner.

For purposes of discussion, embodiments applicable to the field of dentistry (i.e., the treatment of diseases and other conditions that affect the head, neck, teeth and gums) are disclosed. However, the invention is not so limited. It should be readily understood by persons of ordinary skill in the art the applicability of embodiments to other medical fields.

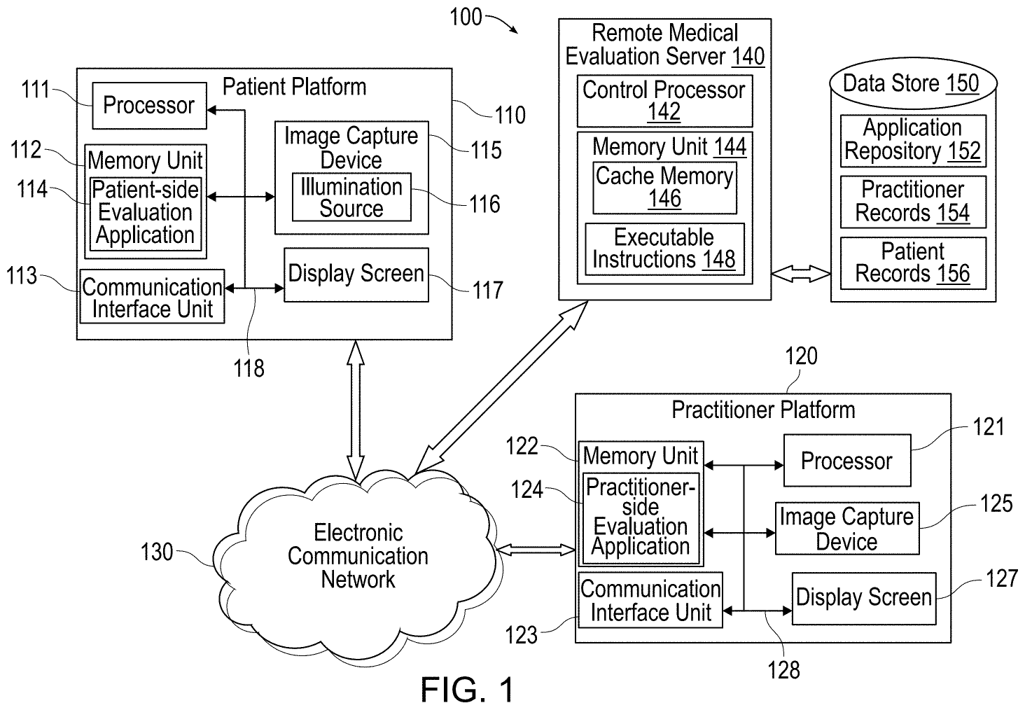

FIG. 1 depicts a remote patient medical evaluation system 100 in accordance with an embodiment of the invention. System 100 includes patient platform 110 that can be in communication with practitioner platform 120 over electronic communication network 130.

Electronic communication network 130 can be the Internet, a local area network, a wide area network, a virtual private network, a wireless area network, or any other suitable configuration of an electronic communication network.

Patient platform 110 can be any type of computing device that includes elements used during the remote patient evaluation session—for example, a handheld computing device such as mobile phone, tablet computing device, personal digital assistant, etc. In accordance with embodiments, other suitable computing devices can include, but are not limited to, a personal computer, a workstation, a thin client computing device, a netbook, a notebook, etc.

Patient platform 110 includes processor 111 that can access executable instructions stored in memory unit 112. When executed by the processor, the executable instructions cause the processor to control operations of the patient platform. The processor is in communication with other elements of the patient platform via control/data bus 118.

Communication interface unit 113 conducts, under the control of processor 111, the input/output transmissions of the patient platform 110. The input/output transmissions can be made by one of several protocols, dependent on the type of computing device. These communication protocols can include, but are not limited to, ethernet, cellular, Bluetooth, Zigbee, and other communication protocols.

In accordance with embodiments, image capture device 115 can operate in visible, ultraviolet, and/or infrared light spectrums. Still or video images can be captured by the image device. These captured images can be stored in memory unit 112, displayed on display screen 117, and/or communicated to an external device across the electronic communication network 130 via communication interface unit 117.

Illumination source 116 can be used during image capture to illuminate the field-of-view of the image device. In accordance with embodiments, the illumination device can generate illumination in a variety of sizes, shapes, and intensities within visible, ultraviolet, and/or infrared light spectrums.

Patient-side evaluation application 114 is a set of executable instructions located in memory unit 112, which when executed cause the patient platform to be used as a remote evaluation and diagnostic tool by the practitioner. The patient-side evaluation application can be an application file pre-installed in the patient platform or obtained as a downloadable file from an application repository.

Practitioner platform 120 can be any type of computing device that includes elements suitable for achieving and implementing the remote patient evaluation session—e.g., a personal computer, a workstation, a thin client computing device, a netbook, a notebook, tablet computer, etc. In accordance with embodiments, other suitable computing devices can include, but are not limited to, mobile phone, tablet computing device, personal digital assistant, etc.

Practitioner platform 120 includes processor 121 that can access executable instructions stored in memory unit 122. When executed by the processor, the executable instructions cause the processor to control operations of the practitioner platform. The processor is in communication with other elements of the practitioner platform via control/data bus 128.

Communication interface unit 123 conducts, under the control of processor 121, the input/output transmissions of the practitioner platform 120. The input/output transmissions can be made by one of several protocols, dependent on the type of computing device. These communication protocols can include, but are not limited to, ethernet, cellular, Bluetooth, Zigbee, and other communication protocols.

In accordance with embodiments, image capture device 125 can capture still or video images. These captured images can be stored in memory unit 122, displayed on display screen 127, and/or communicated to an external device across the electronic communication network 130 via communication interface unit 123.

In accordance with embodiments, communication interface unit 113 and communication interface unit 123 can include hardware and/or software to enable end-to-end encryption/de-encryption protocols to maintain privacy of the communication content.

Practitioner -side evaluation application 124 is a set of executable instructions located in memory unit 122, which when executed cause the practitioner platform to be used by the practitioner to access the patient platform 110 to conduct a remote evaluation and diagnostic session. During the remote session, the practitioner can control various operations of elements of the patient platform 110. The practitioner-side evaluation application 124 can be an application file pre-installed in the practitioner platform or obtained as a downloadable file from an application repository.

Remote medical evaluation server 140 can be in communication with patient platform 110 and practitioner platform 120 across the electronic communication network. The remote medical evaluation server can include a control processor 142 that accesses memory unit 144. Executable instructions 148 when executed by the control processor causes the remote medical server to perform its functions. Cache memory 146 temporarily stores information, data and programs commonly used by the control processor to allow for faster data access and execution by the processor.

Data store 150 can include application repository 152 that contains one or more evaluation applications. Practitioner records 154 can include identification (name, address), practitioners' areas of practice (e.g., dentistry, general medicine, surgery, or other medical specialty), licensing information, billing rates, certification(s), education, and other information. Patient records 156 can include patients' name and address, insurance information, medical history, treatments, pharmacy formulary, etc.

In accordance with embodiments, the patient-side evaluation application 114 and the practitioner-side evaluation application 124 can both be in the same file (pre-installed on respective platforms, or downloadable). Each evaluation application can be adjusted (by an authorized user or system administrator), to enable the features and configurations to perform the respective roles of the patient-side and the practitioner-side evaluation applications. In accordance with embodiments, the application repository can include each of the patient-side evaluation application file, a practitioner-side evaluation application file, and a combined patient/practitioner evaluation application file.

In accordance with embodiments, the practitioner-side evaluation application 124 provides the practitioner with remote access to control components of the patient platform 110, including image capture device 115, illumination source 116, and display device 117. The patient-side evaluation application 114 facilitates the remote access and control of the patient platform by the practitioner.

In accordance with embodiments, control by the practitioner can include remotely adjusting the position, size, intensity, and light spectrum output of the illumination source 116. The practitioner can also control the display screen 117 at pixel and subpixel levels. The practitioner can control the size of the image displayed to a limited portion of the display screen. The practitioner can control the pixels and/or subpixels of the remaining screen portion to create a light bar that illuminates that part of the patient within the field-of-view of the image capture device 115. The light bar can be remotely adjusted by the practitioner to provide illumination in various portions of the light spectrum and at various intensity levels, as needed by the practitioner to perform the evaluation and diagnosis.

The practitioner can control the images captured at the patient platform 110; whether the images are still or video; and which images are provided to the practitioner platform 120 across the electronic communication network 130. In accordance with embodiments, the practitioner can access various image editing features local to the patient platform. These image editing features can be used by the practitioner to graphically annotate the image with adjustable markings. The markings can be used to determine the real-world, physical distances of a patient's features between points identified on the image by the practitioner.

Conventional video conferencing applications lack the ability to provide the practitioner with the remote access and control of the patient platform to adequately evaluate and diagnosis the patient. For example, in the field of dentistry, patients are unable to properly illuminate the mouth. Aiming a camera and a separate light is a challenging task for the patient. As a result, intraoral examination is compromised by shadowing from other portions of anatomy. The cheek, tongue, and maxilla, sometimes mandible, obstruct proper lighting.

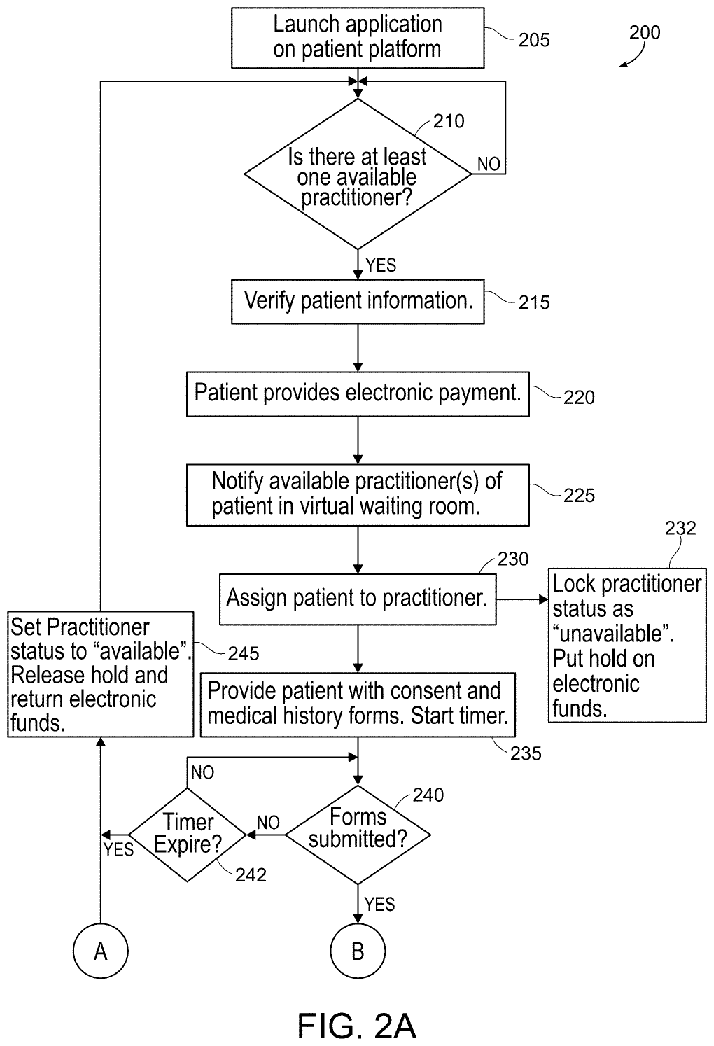

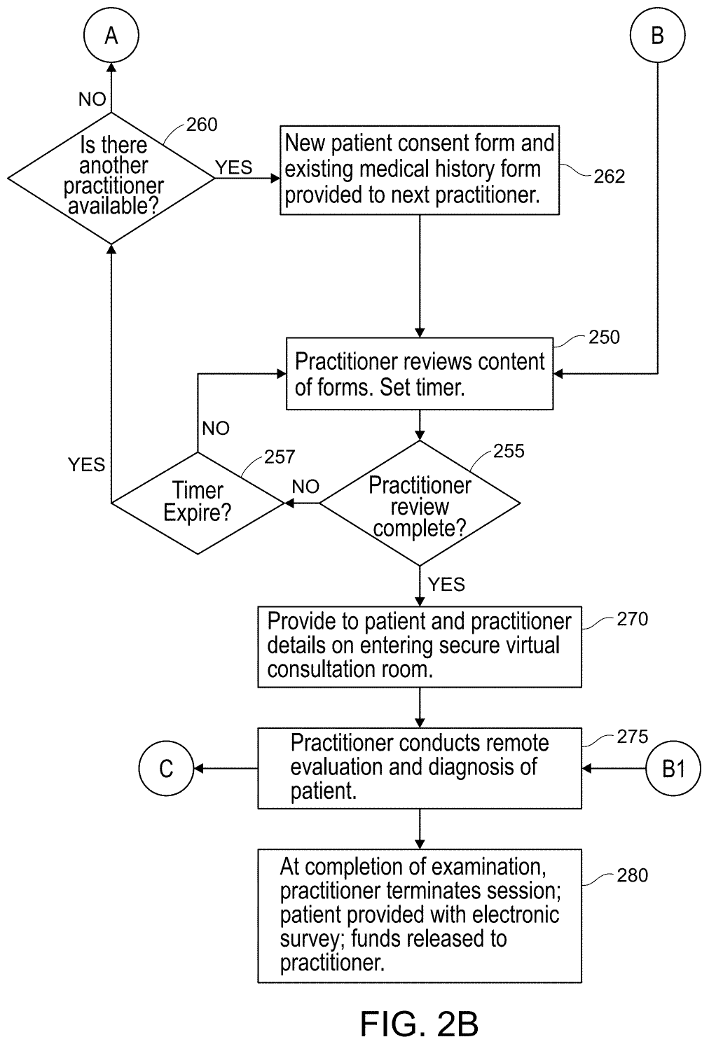

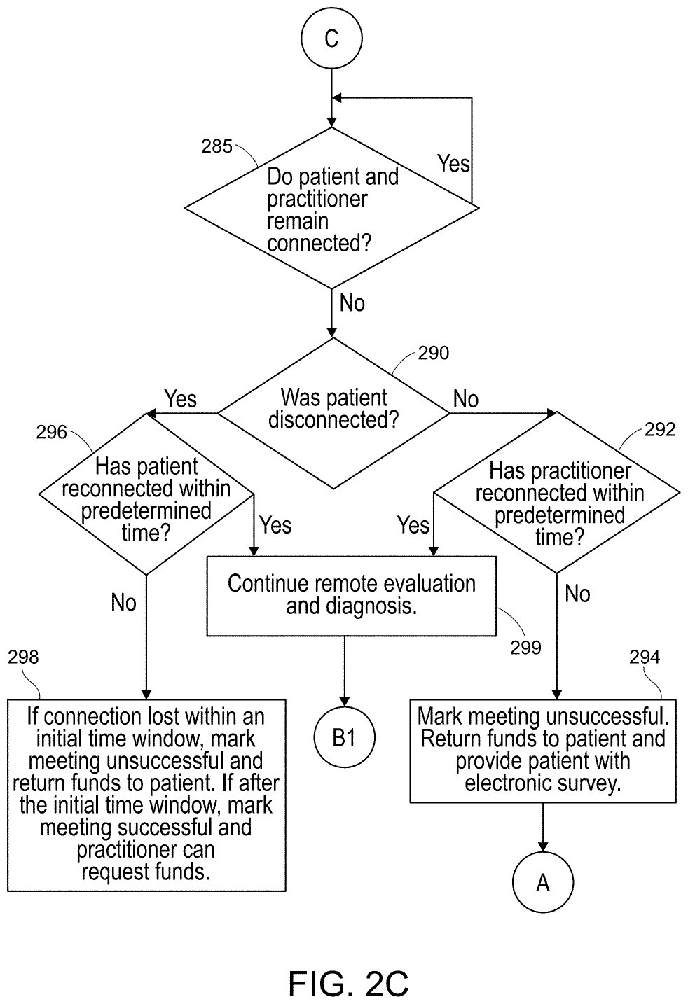

FIGS. 2A-2C depict Remote Patient Medical Evaluation (RPME) process 200 in accordance with embodiments. In accordance with embodiments, RPME process 200 can perform one or more functions including, but not limited to, patient verification and payment authorization, practitioner notification, intake paperwork, and virtual consultation room invite.

After the patient-side application is launched, step 205, a determination is made at the server by the control processor as to whether there are one or more available practitioners, step 210. In accordance with embodiments, this determination can be made by checking a status indicator for individual practitioners in their respective practitioner record. If no practitioner is available, the application loops.

RPME 200 verifies the patient information, step 215, from information entered by the patient on the patient platform. The patient provides electronic payment details, step 220. The payment funds are placed on hold until completion of the virtual visit. Electronic payment can be by credit/debit card, electronic wallet, bank transfer, or other available methods.

At step 225 the available practitioner(s) are notified of a patient. Participating practitioners can be in private practice, thus independent, and unaffiliated with each other. In some instances, group practices can enroll one or more of its members into the system. The system can send a notification to the practitioner—e.g., by text, e-mail, phone, etc.

In accordance with embodiments, the selection order for practitioners to be notified can be done according to a suitability matching metric between the practitioner and the patient, which can be included in data store 150. The criteria determining the suitability matching metric can be done independent of RPME process 200.

Each practitioner can be allocated time to respond before RPME process 200 moves to notify the next most suitable practitioner. After receiving acceptance from the practitioner, the patient is assigned to the practitioner, step 230. The status of the practitioner is locked to “unavailable”, step 232, and the funds are put on hold.

The patient is provided with electronic consent and medical history forms to be completed for intake as a new or existing patient, step 235. A timing loop is entered, steps 240 -242, during which the patient needs to submit the forms. If the forms are not submitted prior to expiration of the timer, RPME process 200 sets the practitioner status to “available”, releases the hold and returns the electronic funds, step 245.

If the forms are timely submitted, the selected practitioner reviews the content of the forms, step 250. A timing loop is entered, steps 255 -257, during which the practitioner needs to review the forms' content. If the practitioner has not completed the review prior to expiration of the timer, RPME process 200 makes a determination as to the availability of another practitioner, step 260. If there is another practitioner, that next practitioner is provided the consent and medical history forms, step 262. This newly selected practitioner is then given the opportunity to review the forms, step 250. If no other practitioner is available, RPME process 200 returns to step 245.

Upon completion of the practitioner review, both the practitioner and patient are provided details on entering the secure, virtual consultation room, step 270. Embodiments of the RPME system are not limited to the nature of the virtual consultation room, which can be implemented by many different providers. The practitioner conducts the remote evaluation and diagnosis of the patient, step 275.

At the completion of the examination, step 280, the practitioner terminates the session, the electronic funds are released to the practitioner, and the patient is provided with an electronic survey.

While the remote evaluation and diagnosis is ongoing (step 270), the connectivity of both the patient and practitioner to the virtual examination room is monitored, step 285. If the connection is maintained, the monitoring loops onto itself. Once the connection is lost, a determination is made to whether the patient disconnected, step 290. If the patient reconnects within a predetermined time limit, step 296, the remote evaluation and diagnosis continues, step 299.

If the patient lost connection within an initial time window from entering the virtual consultation room, step 286, the meeting is marked unsuccessful and the electronic funds are returned. If the patient connection is lost after this initial time window, the meeting is marked successful and the practitioner can request the electronic funds. This initial time window can be preset by the system, or negotiated in advance as a practitioner criterion, or a patient criterion; and stored in the practitioner record 154 or the patient record 156.

If at step 290 the patient remains connected, a determination is made as to whether the practitioner reconnects within a predetermined time limit, step 292. If the practitioner timely reconnects, the remote evaluation and diagnosis continues, step 299. If the practitioner has not timely reconnected, step 294, the meeting is marked unsuccessful, the funds are returned, and the patient is provided an electronic survey. RPME process then returns to step 245.

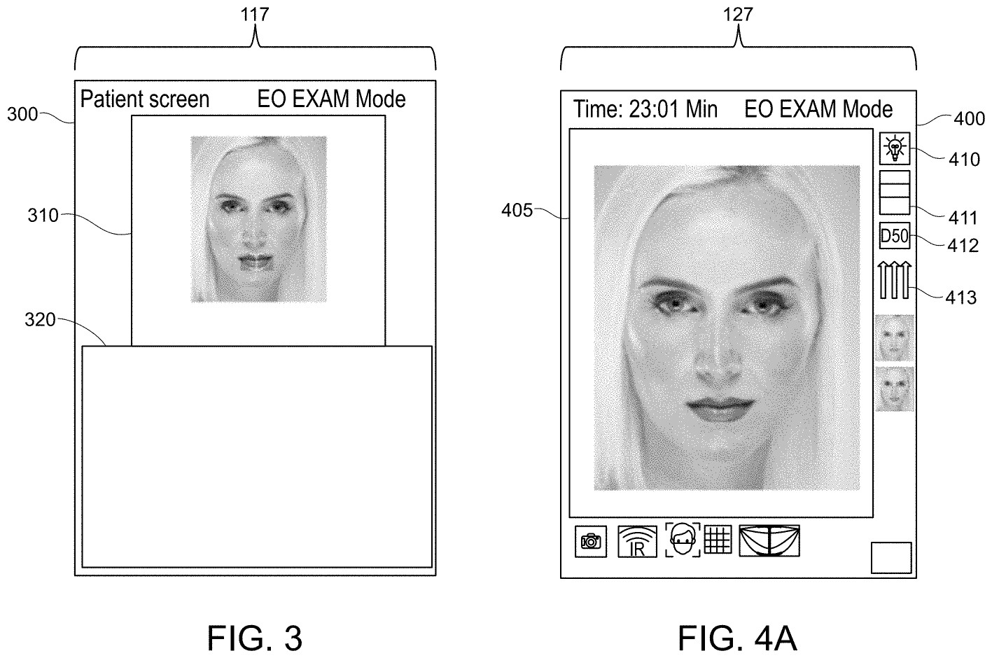

FIG. 3 depicts a patient view of interactive display 300 in accordance with embodiments. Display 300 appears on the display screen 117 of the patient platform 110. In accordance with embodiments, display screen 117 is divisible under the control of the practitioner by communications from the practitioner platform 120. Display screen 117 can be a forward-facing screen (i.e., facing the patient). Window 310 presents an image within the field-of-view of image capture device 115. The patient can use this image to self-align the field-of-view to the region being evaluated by the practitioner. The practitioner can adjust the positioning, type, and size of this image (silhouette) to further align the patient.

As noted above, embodiments pertaining to the field of dentistry will be discussed. However, the invention is not so limited and can be readily tailored to meet the specialized diagnostic needs and tools of other medical specialties.

Window 320 is a light bar used to illuminate the region of interest. The size, illumination intensity, and light spectrum of the light bar are controllable by commands from the practitioner platform. In accordance with embodiments, the light bar can be controlled to form a perimeter around the field-of-view window 310.

Once the patient has properly aligned window 310 for the practitioner's examination, under the practitioner's control the whole of display screen 117 can be made to be the light bar 320. The bar illuminates the patient, and helps compensate for ambient lighting conditions. In accordance with embodiments, light bar 320 can be remotely controlled by the practitioner.

The color temperature of the light bar can be controlled from the practitioner platform. In accordance with embodiments, a D50 light source best approximates natural day light. For example, a base lighting source can have a red, green, blue (RGB) pixel value of R255, G246, B237. These values can be varied by the examining clinician to aid in illuminating the examination region.

In accordance with embodiments, adjustment to the light bar can be made to achieve visible, ultraviolet, and/or infrared light spectrums For example, the RGB values can be adjusted to react with fluorescent markers. The dentist can provide a prescription for a fluorescent rinse that responds to a known wavelength of light. The fluorescent rinse can be selected to detect disease (such as oral cancer). During the remote examination, the RGB value can be changed to a wavelength known photoactivates with the fluorescent dye. The practitioner can also change the illumination light spectrum to exacerbate reflective properties of certain pathologies for remote detection and diagnosis. Clinicians may also change the illumination light spectrum to exacerbate the reflective properties of certain pathologies.

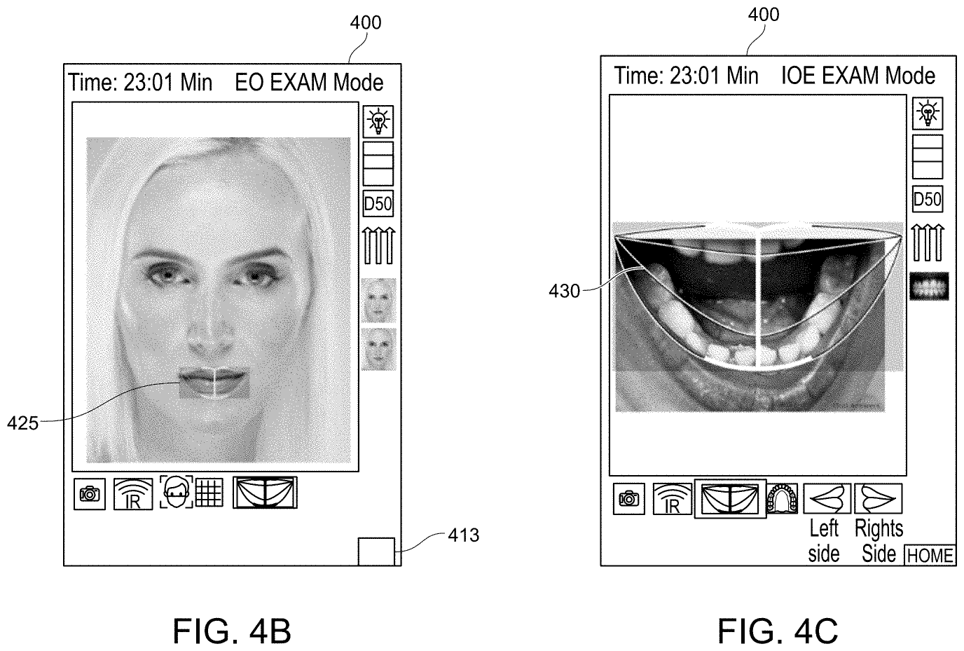

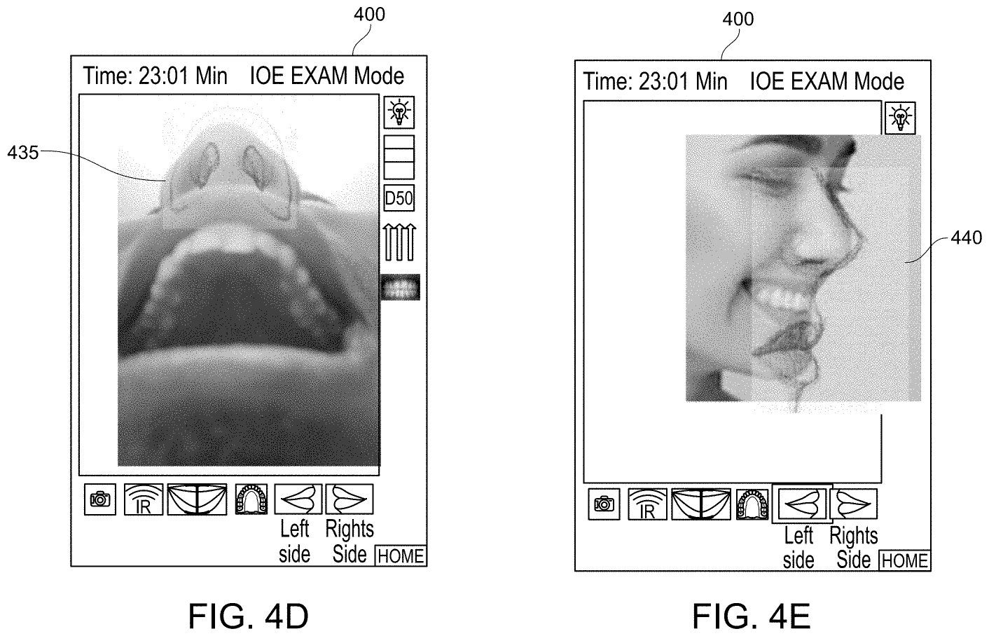

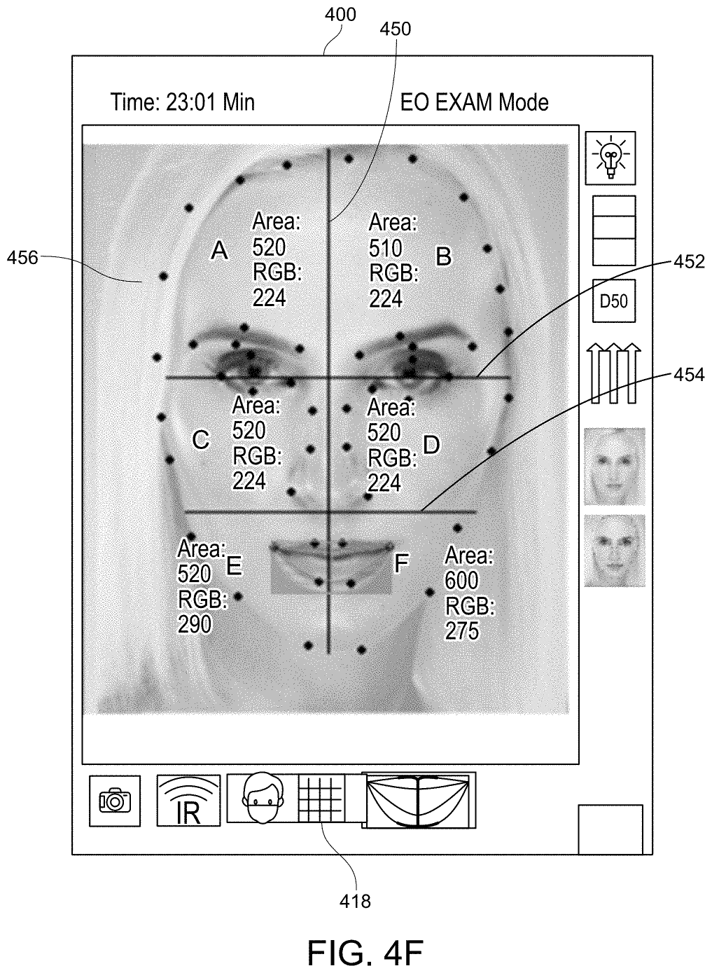

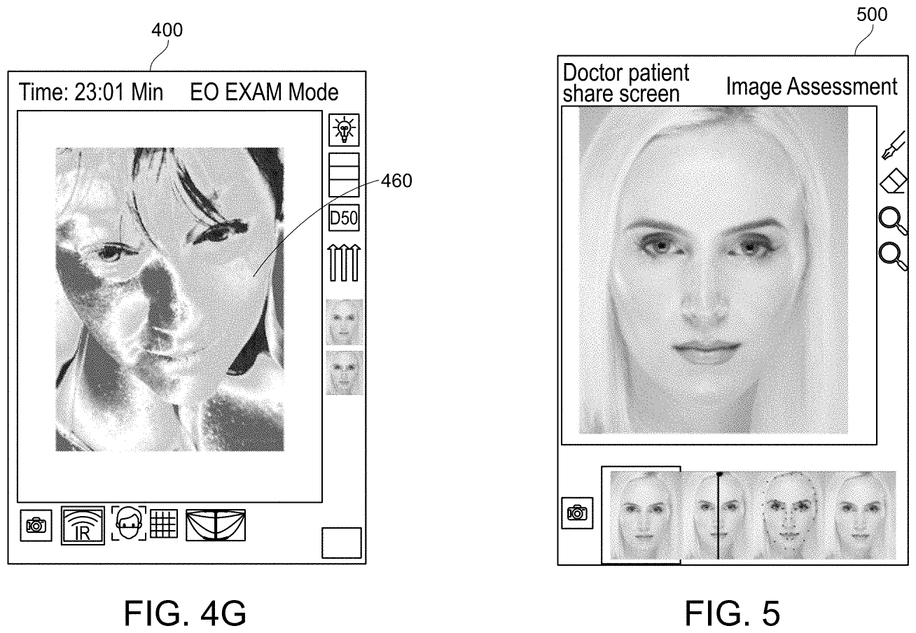

FIGS. 4A-4G depict practitioner views of interactive display 400 in accordance with embodiments. Display 400 appears on the display screen 127 of the practitioner platform 120. Arrayed on display 400 are interactive buttons 410-421 that are selectable by the practitioner to remotely control the function of components at the patient platform 110. Although illustrated along the perimeter of display 400, the configuration, location, and function of the buttons can be adjusted to personalize the practitioner platform.

Activating button 410 can change the intensity of the light bar 320. Successive activations can toggle the intensity to predetermined values. Activation of button 411 can toggle the location of the light bar within display 300. The size of the light bar can be controlled by activation of button 412.

The color temperature, and/or light spectrum of the light bar can be adjusted by activation of button 413. Interactive display 400 provides the practitioner with the ability to remotely control the illumination produced at the patient platform in a manner to facilitate the remote examination and diagnosis.

Embodying systems and methods provide the practitioner with the ability to instruct the patient on where to orientate their image on the display. Conventional video conferencing systems lack an initial orientation and/or positioning reference. This lack of reference makes it difficult for a practitioner to give instructions to the patient for reorienting the view(s). Patients also have difficulty in satisfying the practitioner's instruction on how to position themselves to provide the practitioner with the angles and perspectives needed for the medical evaluation and diagnosis.

The ability to accurately position a patient within the field-of=view of the image capture device is important in the practice of dentistry. Embodying systems and methods provide the practitioner to remotely superimpose on the display screen 117 at the patient platform a marker. The practitioner can view the marker 425 (FIG. 4B) on their local display screen 127. The marker position can be controlled at the practitioner platform, causing it to reposition on the patient platform display screen.

Positioning the marker and guiding the patient to position themselves within boundaries displayed on their screen allows for alignment of the patient. In accordance with embodiments, different markers can be remotely superimposed for different imaging requirements. Patient morphology varies, as do the devices being used as the patient platform 110. The superimposed positioning marker accommodates for these differences of anatomy and technology.

With reference to FIG. 4B, a dentistry practitioner can superimpose a marker in the image of lips 425. This superimposed image can then appear on the patient interactive display 300. The patient can then orientate themself. Once properly orientated, the practitioner can then activate camera button 414 to capture a properly registered facial image. Once a properly registered image is captured, prior and/or subsequent registered images can be used by the practitioner for comparative and measurement purposes.

Having a reference marker superimposed on the patient interactive display allows for clearer and more efficient interaction between the practitioner and patient during the evaluation. For example, a dentist can request that the patient to lift their chin to examine the lower dentition; to lower their chin to examine the maxillary dentition; and to turn their chin to the left or right to examine additional surfaces.

With reference to FIG. 4C, after alignment the patient is instructed to tilt their head down to obtain a view of the lower dentition. An annotation marker 430 can be used to register features of the image. With reference to FIG. 4D, the practitioner can superimpose a nostril stencil 435 at the practitioner interactive display 400. The nostril stencil can be viewed by the patient on the patient interactive display 300. After the patient aligns their nostrils to the nostril stencil, an image of the maxillary (upper teeth) dentition can be registered by control of the image capture device 115 from the practitioner platform 120.

With reference to FIG. 4E, the practitioner can superimpose a side profile stencil 440 at the practitioner interactive display 400. The patient is instructed to rotate the camera 90 degrees to the left and turn their head to the right to align with the stenciled side profile. After the patient retracts their lips (by smiling or with their fingers), an image of the left-side profile of the dentition and face can be captured by control of the image capture device 115 from the practitioner platform 120. The right-side profile of the dentition and face can be similarly captured.

Embodying systems and methods can be used to evaluate infection and/or inflammation. Infections and inflammations are often accompanied with a vascular response. The vascular response can be vascular dilation, which can manifest in a change in the coloration of the outer skin surface. For example, coloration changes can lead to red inflammation, but blue and/or yellow bruising can also appear in an affected area.

Embodiments can use asynchronous images, or synchronous videos to derive RGB pixel data of the captured images. The derived RGB data can be used to compare RGB average scores with contralateral sides of the patient's body; or with RGB values from previously obtained and stored images of the same location. For example, a red score can be generated based on the value of the red subpixel. Pixels having high red subpixel values can be graphically linked by levels to form contours of an erythema map indicating an inflammation boundary.

With reference to FIG. 4F, once the patient is aligned and registered to the screen the practitioner can activate coordinate recognition button 418 to generate a superimposed grid on the patient image. When superimposed on the face, this grid can be used by a facial recognition method can be used to generate a facial outline 456.

The face can be divided into sectors along a midline 450, a sub-nasal line 452, and an intercanthal line 454. The RGB values of these sectors are displayed. The practitioner can compare contralateral RGB data to evaluate the patient's condition. Because illumination of the subject is uniform, an accurate comparison can be made. An exaggerated red spectrum response can indicate inflammation and/or infection, where red shift indicates disease. In some cases, comparison can be made between the current image and prior stored images.

The visible light's red spectrum response can be used to generate a red map that indicates local variation in surface temperature. This red map can be used to identify one or more regions of potential infection or inflammation by comparison to contralateral structures.

The vascular response to infection and/or inflammation can induce swelling. This swelling can be undetectable during a conventional videoconferencing session. Embodying alignment, tools, analysis of orientated gradients (as disclosed with reference to FIG. 4F) can be applied to analyzing captured still and video images. In accordance with embodiments, this analysis can:

Compare facial dimensions with the contralateral side of the face;

Compare facial areas-volume with the contralateral side of the face; and

Identify the facial midline composed of the center of the philtrum and the center point of the intercanthal line.

In accordance with embodiments, application of these tools can be used to obtain measurements for comparative purpose. These measurements can include:

The vertical distance from the goniao angle to the palpebral outboard angle;

The horizontal distance to the corner (i.e., distance from external angle of the mouth to the bottom edge of the earlobe);

The horizontal distance to the symphasis (i.e., from the bottom edge of the earlobe to the midpoint of the symphasis).

The vascular response from infection and/or inflammation can also result in localized thermal gradients between the affected area and surrounding and/or contralateral anatomy. In accordance with embodiments, from the practitioner platform 120 a practitioner can remotely adjust the light spectrum of the image capture device 117 and the illumination source 116 at the patient platform 110 to operate in the infrared spectrum.

With reference to FIG. 4G, facial heat map 460 can be generated from the IR illumination image. The depicted image of the heat map can show local variations in surface temperature by changing the intensity of the image (e.g., darker areas being cooler than brighter areas). This facial heat map can indicate isothermal regions distinct from other heterogenous regions. A practitioner can identify regions of infection and/or inflammation by comparison with contralateral structures.

FIG. 5 depicts collaborative interactive display 500 with dual accessibility to both the patient and the practitioner in accordance with embodiments. Collaborative interactive display 500 can be viewed by both the patient (on display screen 117) and practitioner (on display screen 127) so that patient and practitioner can engage in an interactive annotation session to discuss symptoms and/or findings. In accordance with embodiments, collaborative interactive display 500 provides for patient and doctor collaborative communication via an audio/video channel with the ability to make markings and annotations on the registered images. Using the collaborative display, a patient can identify areas of concern prior to examination, and a practitioner can convey findings to the patient.

In accordance with embodiments, patient and/or practitioner can manipulate the size of the images. The annotated images captured by pressing the camera button. The captured image can then be transferred from/to the patient and/or practitioner platform.

In accordance with an embodiment of the invention, a computer program application stored in non-volatile memory or computer-readable medium (e.g., register memory, processor cache, RAM, ROM, hard drive, flash memory, CD ROM, magnetic media, etc.) may include code or executable instructions that when executed may instruct or cause a controller or processor to perform methods discussed herein such as a method for conducting a medical evaluation and diagnosis of a patient through remote control of a communication platform local to the patient.

The computer-readable medium may be a non-transitory computer-readable media including all forms and types of memory and all computer-readable media except for a transitory, propagating signal. In one implementation, the non-volatile memory or computer-readable medium may be external memory.

Although specific hardware and data configurations have been described herein, note that any number of other configurations may be provided in accordance with embodiments of the invention. Thus, while there have been shown, described, and pointed out fundamental novel features of the invention as applied to several embodiments, it will be understood that various omissions, substitutions, and changes in the form and details of the illustrated embodiments, and in their operation, may be made by those skilled in the art without departing from the spirit and scope of the invention. Substitutions of elements from one embodiment to another are also fully intended and contemplated. The invention is defined solely with regard to the claims appended hereto, and equivalents of the recitations therein.

Claims:

1. A method for remote medical evaluation of a patient, the method comprising:
providing a patient-side application to a patient platform;
providing a practitioner-side application to a practitioner platform;
establishing an electronic communication connection between the patient platform and the practitioner platform;
transmitting electronic command signals from the practitioner platform to the patient platform to remotely control one or more components of the patient platform;
receiving at the practitioner platform information obtained from operation of the one or more remotely controlled patient platform components; and
providing on the practitioner platform the obtained information to a medical practitioner.

2. The method of claim 1, including:
observing in about real-time at the practitioner platform images captured by a patient platform image capture device, the images captured in accordance with one or more of the transmitted electronic command signals;
introducing from the practitioner platform one or more adjustable graphical annotation markings on an image being displayed at the patient platform;
instructing the patient to align themself with the annotation markings;
obtaining at the practitioner platform real-world physical distance measurements of patient features located between points included in the annotation markings; and
providing on the practitioner platform the obtained real-world physical distance measurements to a medical practitioner.

3. The method of claim 1, including:
controlling from the practitioner platform a light bar being generated on a display screen of the patient platform, the control including at least one of an illumination intensity, light spectrum, color temperature, geometric size and shape, or position of the light bar;
introducing from the practitioner platform graphical annotation markings on an image being displayed at the patient platform;

4. The method of claim 1, including the transmitting of electronic command signals from the practitioner platform being initiated by activation of one or more buttons displayed on an interactive display at the practitioner platform.

5. The method of claim 1, including:
introducing from the practitioner platform graphical annotation markings on an image being displayed at the patient platform;
instructing the patient to algin themself with the graphical annotation markings; and
remotely controlling from the practitioner platform an image capture device at the patient platform to obtain an image of the aligned patient.

6. The method of claim 5, including at least one of:
the graphical annotation is an image of lips and the obtained image is of lower dentition;
the graphical annotation is a nostril stencil and the obtained image is of maxillary dentition; or
the graphical annotation is a side profile stencil and the obtained image is a profile of the patient's dentition and face; or

7. The method of claim 1, wherein the obtained information is an image, the method including:
generating a grid superimposed on the obtained image;
derive from the obtained image contributions to a red-blue-green (RGB) pixel data from individual red subpixels, green subpixels, and blue subpixels; and
comparing the RGB to contralateral or previously obtained images of the patient.

8. The method of claim 1, wherein the obtained information is an image, the method including:
applying a facial recognition technique to the obtained image to determine facial dimensions; and
comparing the facial dimensions to contralateral or previously obtained images of the patient.

9. The method of claim 1, wherein the obtained information is an image, the method including:
instructing from the practitioner platform an image capture device at the patient platform to operate within an infrared spectrum;
generating a heat map from an infrared image, the heat map indicating local variation in surface temperature; and
identifying one or more regions of potential infection or inflammation by comparison to contralateral structures.

10. The method of claim 1, wherein the obtained information is an image, the method including:
instructing from the practitioner platform an image capture device at the patient platform to operate within a visible light spectrum;
generating a red map from a visible light image, the red map indicating local variation in surface temperature; and
identifying one or more regions of potential vascular dilation, infection, or inflammation by comparison to contralateral structures.

11. The method of claim 1, wherein the obtained information is an image, the method including:
displaying on a practitioner platform display screen and a patient platform display screen a shared image; and
providing abilities to annotate the shared image at both the practitioner platform and the patient platform.

12. A non-transitory computer readable medium having stored thereon instructions which when executed by a processor cause the processor to perform the method of:
providing a patient-side application to a patient platform;
providing a practitioner-side application to a practitioner platform;
establishing an electronic communication connection between the patient platform and the practitioner platform;
transmitting electronic command signals from the practitioner platform to the patient platform to remotely control one or more components of the patient platform;
receiving at the practitioner platform information obtained from operation of the one or more remotely controlled patient platform components; and
providing on the practitioner platform the obtained information to a medical practitioner.

13. The computer readable medium of claim 12, further including executable instructions to cause the processor to perform the method including:
observing in about real-time at the practitioner platform images captured by a patient platform image capture device, the images captured in accordance with one or more of the transmitted electronic command signals;
introducing from the practitioner platform graphical annotation markings on an image being displayed at the patient platform;
instructing the patient to align themself with the annotation markings;
obtaining at the practitioner platform real-world physical distance measurements of patient features located between points included in the annotation markings; and
providing on the practitioner platform the obtained real-world physical distance measurements to a medical practitioner.

14. The computer readable medium of claim 12, further including executable instructions to cause the processor to perform the method including:
controlling from the practitioner platform a light bar being generated on a display screen of the patient platform, the control including at least one of an illumination intensity, light spectrum, color temperature, geometric size and shape, or position of the light bar;
introducing from the practitioner platform graphical annotation markings on an image being displayed at the patient platform;

15. The computer readable medium of claim 12, further including executable instructions to cause the processor to perform the method including the transmitting of electronic command signals from the practitioner platform being initiated by activation of one or more buttons displayed on an interactive display at the practitioner platform.

16. The computer readable medium of claim 12, further including executable instructions to cause the processor to perform the method including:
introducing from the practitioner platform graphical annotation markings on an image being displayed at the patient platform;
instructing the patient to algin themself with the graphical annotation markings; and
remotely controlling from the practitioner platform an image capture device at the patient platform to obtain an image of the aligned patient.

17. The computer readable medium of claim 16, further including executable instructions to cause the processor to perform the method including at least one of:
the graphical annotation is an image of lips and the obtained image is of lower dentition;
the graphical annotation is a nostril stencil and the obtained image is of maxillary dentition; or
the graphical annotation is a side profile stencil and the obtained image is a profile of the patient's dentition and face; or

18. The computer readable medium of claim 12, further including executable instructions to cause the processor to perform the method, wherein the obtained information is an image, the method including:
generating a grid superimposed on the obtained image;
derive from the obtained image contributions to a red-blue-green (RGB) pixel data from individual red subpixels, green subpixels, and blue subpixels; and
comparing the RGB to contralateral or previously obtained images of the patient.

19. The computer readable medium of claim 12, further including executable instructions to cause the processor to perform the method, wherein the obtained information is an image, the method including:
applying a facial recognition technique to the obtained image to determine facial dimensions; and
comparing the facial dimensions to contralateral or previously obtained images of the patient.

20. The computer readable medium of claim 12, further including executable instructions to cause the processor to perform the method, wherein the obtained information is an image, the method including:
instructing from the practitioner platform an image capture device at the patient platform to operate within an infrared spectrum;
generating a heat map from an infrared image, the heat map indicating local variation in surface temperature; and
identifying one or more regions of potential infection or inflammation by comparison to contralateral structures.

21. The computer readable medium of claim 12, further including executable instructions to cause the processor to perform the method, wherein the obtained information is an image, the method including:
instructing from the practitioner platform an image capture device at the patient platform to operate within a visible light spectrum;
generating a red map from a visible light image, the red map indicating local variation in surface temperature; and
identifying one or more regions of potential vascular dilation, infection, or inflammation by comparison to contralateral structures.

22. The computer readable medium of claim 12, further including executable instructions to cause the processor to perform the method, wherein the obtained information is an image, the method including:
displaying on a practitioner platform display screen and a patient platform display screen a shared image; and
providing abilities to annotate the shared image at both the practitioner platform and the patient platform.
